(19) United States
(12) Patent Application Publication
(10) Pub. No.: US 2020/0376002 A1
(43) Pub. Date: Dec. 3, 2020

(54) Injectable Hormone Formulations for Estrous Cycle Control in Mammals, its Manufacturing Process, Estrous Cycle Control and Puberty Triggering Methods, and Pregnancy Enhancement in Mammals

(71) Applicant: Applied Technologies Consultants Inc., Miami Beach, FL (US)

(72) Inventor: Claudio Carlos PAOLAZZI, Buenos Aires (AR)

(73) Assignee: Applied Technologies Consultants Inc., Miami Beach, FL (US)

(21) Appl. No.: 16/426,639

(22) Filed: May 30, 2019

(51) Int. Cl. A61K 31/57; A61K 31/565; A61K 47/44; A61K 47/26; A61K 47/10; A61K 47/14; A61K 9/50; A61K 31/5575; A61P 15/08

(52) U.S. Cl. CPC A61K 31/57; A61K 31/565; A61K 47/44; A61K 47/26; A61P 15/08; A61K 47/14; A61K 9/5031; A61K 31/5575; A61K 47/10

(57) ABSTRACT

Hormone Injectable Formulations for controlling the estrous cycle in mammals, triggering puberty and improving pregnancy in mammals, including a hormone, a Sucrose polymer, organic solvents, vegetable oils, oxyethylene polymers and excipients, where the formulation forms porous microspheres when in contact with a water-based liquid medium, and where the formulation is a single-phase clear liquid. The hormone can be Progesterone or its derivatives, alone or in combination with Estradiol.

This invention refers to a Hormone Injectable Formulation for Estrous Cycle Control in Mammals and its development process. In particular, the formulation includes a hormone, a modified disaccharide, organic solvents, vegetable oils, oxyethylene polymers and excipients, where the Formulation forms porous microspheres when in contact with a water-based liquid medium, and where the Formulation is a single-phase clear liquid. The hormone can be, without limitations, Progesterone or its derivatives.

### BACKGROUND

Progestagenic agents have long been used in the Pharmaceutical industry both to control estrous cycles of animals which bear a commercial interest for people, and to maintain a risky pregnancy and increase corpus luteum effect in bovine pregnancies. It has been discovered that the Progesterone hormone (hereinafter, P4), or similar synthetic elements, is capable of preventing ovulation in different mammals, stopping follicular ovulation. Research shows that when P4 decreases to basal levels, within a reasonable period of time that is in line with a specific and non-ageing follicular growth (or a dominant follicle in certain mammals, such as cows), the result is a viable ovulation that can be triggered and/or increased by the effect of ovulation triggers, such as Estrogen (E2) or Gonadotropin-releasing Hormones (GnRH). Prostaglandins (such as D or D,L-Cloprostenol) lyse the corpus luteum and contribute to the reduction of endogenous Progesterone levels. Considering all of these parameters and hormones, it is possible to have complete control over the estrous cycle of certain mammals to synchronise ovulations, and, as a result, perform timely inseminations that will allow us to group calving. These strategies were applied in cows and sows, and they showed great potential in goats, horses, and human beings. This description details references and tests performed on bovine livestock.

As mentioned above, in cows, P4 may inhibit ovulations provided it is administered constantly for a period of time long enough to allow growing follicles to mature. To this end, different hormone presentations have been produced and commercialized for a long time. These include silicone subcutaneous implants, polyurethane intra-vaginal sponges, and the widely used silicone intra-vaginal devices (SID), which are impregnated with Progesterone. The latter are the most successful devices in the market, even though they are widely known for being both difficult to introduce and uncomfortable for the animal. As an alternative to the methods described above, P4 release injectable systems have been developed, but they have not proved effective enough to replace the Device.

To date, injectable Progesterone has not succeeded for several reasons. One of the most significant reasons is that the Device can be removed, which overrides the Progesterone input at the appropriate moment for ovulation. Another reason is that they do not reach 7 or 8 days of constant release, therefore, they do not guarantee the hormone decrease to the basal levels needed for ovulation. Moreover, they are metabolic dependent, which impedes adaptation to an animal of higher or lower demand or capacity to absorb the Progestagenic agent, as in the case of cows in nutritional anestrus, postpartum or high dairy production. However, injectable systems are more hygienic; reduce management time, and the need for additional workforce to help in herd management. Furthermore, regarding animal health, injections prevent animals from suffering traumas for having an alien item inside their vaginas, which may cause local infections or serve as the route of entry of parasites. According to the above mentioned, it is evident that the injectable systems need to produce an in situ mechanism in the animal, regardless the occasional metabolism but dependant on the volume administered.

Patent Document US 2014/0335193 discloses PVP microspheres.

Patent Document US 2015/0148323 discloses Progesterone tablets for menopause treatment and Progesterone supplements in risky pregnancies. The article “Indian Journal of Animal Reproduction 33 (2): December 2012” discloses the use of Progesterone depot in a treatment scheme in rodents.

Patent Documents WO2017106618 and WO2017105512 describe an extented release system for estrus cycle control in mammals via the administration of Progesterone. The research introduces an heterogeneous gel-like emulsion/suspension formulation presented in pre-filled syringes, which limits the appropriate dosage and administration of the product for young cows categories and mammals from other species that requiere different Progesterone concentrations or a different Hormone/Vehicle ratio.

Reproduction Manuals state that the main purpose of a cost-effective livestock industry is to breed one calf per cow on a yearly basis. This is the criterion defined because the longer a cow takes to get pregnant, the longer it will take for the calf to be born, the less fodder available there will be, and the less the sale lot of weaned calves will weigh. In the first place, to achieve this purpose, 100% of the cows destined for reproduction must get pregnant. This is not always possible due to health constraints, fertility issues or bull availability, among others. Furthermore, there is also the issue of time that does not provide a broad margin for handling pregnancy of every cow in a calendar year. In other words, we can describe all the events that interact with pregnancy in terms of time, as follows:

a) A year includes 365 days;
b) 273 days correspond to the gestation period;
c) 45 days correspond to the postpartum anestrus;
d) 10 days correspond to treatment period of high reproductive efficiency, such as artificial insemination (AI) or Artificial Insemination at Fixed Time (AIFT); and
e) 30 days correspond to standby time until detecting pregnancy through ultrasound.

Specifically, if pregnancy does not occur in 100% cows during the 40-45 days period, as described in Items d) and e), the purpose is unattainable.

Regardless these considerations, the global pregnancy average after AIFT is 50%.

Although this may seem essential to improve livestock reproduction, AI and AIFT do not account for more than 10% of every female cow in the world. This implies that the remaining 90% still rely on and proceed with natural matings. Predictably, natural pregnancy depends on the same time factors as cow physiology does, apart from the “number of bulls available” factor. For instance, a British breed bull can only mate with 30 cows per month, on a seasonal basis.

Regarding natural mating, mating seasons are established as the period in which a bull copulates with the cows so as to inseminate them. This period is determined by the months of fodder availability, as it is essential for the first months of both calf and mother, since birth until weaning. Beginning at the time bulls and cows mate, this period accounts for 120 days, approximately.

Mating season can be technically divided into three months, also known as calving beginning, middle and end, according to the insemination time during the 120 day period of reproductive service or natural mating. If we support the idea of a sustainable livestock farming mentioned before, the greatest number of cows must be inseminated at the beginning of the mating season.

Naturally, after a 5-day anestrus (prepuberal, nutritional and postpartum), the first follicular wave appears, originating a dominant follicle around Day 11. However, the onset of ovulation in this period is very low, about 10% and 20 days after the beginning of lactation. Follicles that succeed in ovulation are followed by a brief luteal phase, as contractions of the uterus trigger Prostaglandins release.

This invention reduces follicular activation times, regardless the age, breed and/or category of the cow. It also helps reduce time in nutritional anestrus, prepuberal anestrus, and postpartum anestrus. Therefore, it is possible to achieve more ovulations and pregnancies irrespective of the reproduction methodology selected, whether AI, AIFT or natural mating with bulls.

### BRIEF DESCRIPTION OF THE INVENTION

A hormone injectable formulation is provided for controlling the estrous cycle in mammals, including a hormone, an esterified carbohydrate, organic solvents, vegetable oils, oxyethylene polymers and excipients, where the formulation forms an intra-parenteral depot when in contact with a water-based liquid medium, and (the depot) contains permeable porous microspheres. This allows for two release phases: a continuous phase for follicular growth; and an accelerated phase for achieving ovulation. Here, the Formulation is a single-phase clear liquid. In a preferred testing, the hormone is Progesterone, Estradiol Benzoate, Estradiol Cypionate, D-Cloprostenol Sodium, DL-Cloprostenol Sodium, Deslorelin Acetate, Equine Corionic Gonadotrophin, Follicle-stimulating Hormone, Luteinizing Hormone, Buserelin Acetate and/or Oxytocin. In a more preferred testing, the hormone is Progesterone, Medroxyprogesterone, 17-Hydroxyprogesterone, Medroxyprogesteronte Acetate, Megestrol Acetate, Algesterone, Gestonorone, Ethisterone, Norgestomet, Chlormadinone, Altrenogest, Nomegestrol and/or Cyproterone Acetate.

In a preferred testing, the esterified carbohydrate is Sucrose Acetate Isobutyrate (SAIB), Cellulose Acetate Butyrate (CAB), Cellulose Acetate Propionate (CAP), or biodegradable polymers, such as Polycarpolactone, Polylactic Acid and/or Polyactic-co-Glycolic Acid (P1GA).

In a preferred testing, the organic solvent is vegetable triacetin, isopropyl myristrate, ethyl n-methyl-pyrrolidone oleate, PEG400, Tween 20, Mygliol 812, Imwitor 375, Acetyl Tributyl Citrate (ATBC), Dimethylacetamide (DMA), Glycerol formal, Solutol HS15, Benzyl Benzoate, Benzyl Alcohol and/or Ethanol.

In a preferred testing, the vegetable oil is sunflower oil, castor oil, soybean oil, corn oil and/or sesame oil.

In a preferred testing, the oxyethylene polymer is Poloxamer 188 and/or Poloxamer 127.

In a Formulation preferred testing, it includes Sucrose Acetate Isobutyrate, Ethanol, Benzyl Benzoate, Benzyl Alcohol, vegetable oil, and at least one oxyethylene polymer, selected from the group consisting of Poloxamer 188 or Poloxamer 127. As a beneficial option, this Formulation also includes Estradiol. In a more preferred testing, the ratio between SAIB and at least one oxyethylene polymer lies in 10:2 to 10:0.1, specifically, 10:1 a 10:0.4, though the preferred ratios are 10:1, 10:0.925, 10:0.52 and 10:0.41. In a preferred aspect of this invention, the ratio between SAIB and the Poloxamer is 10:1 or 10:0.925.

In a more preferred testing, the formulation is 50-120 mg/ml Sucrose Acetate Isobutyrate, 900-190 mg/ml Ethanol, 850-190 mg/ml Benzyl Benzoate, 850-190 mg/ml Benzyl Alcohol, 850-170 mg/ml vegetable oil, and 50-3 mg/ml of at least one oxyethylene polymer, either Poloxamer 188 or Poloxamer 127.

In another more preferred testing, the formulation also includes Estradiol. For instance, the formulation contains 50-80 mg/ml Progesterone, 0.040-0.025 mg/ml Estradiol, and 50-120 mg/ml Sucrose Acetate Isobutyrate, 900-190 mg/ml Ethanol, 850-190 mg/ml Benzyl Benzoate, 850-190 mg/ml Benzyl Alcohol, 850-170 mg/ml vegetable oil, and 50-3 mg/ml of at least one oxyethylene polymer, selected from the group consisting of Poloxamer 188 or Poloxamer 127.

The formulation presents a 2-6 cP viscosity at 20° C., preferably 5 cP viscosity at 20° C.

The formulation can be administered as multi-dose to several mammals, for instance, cows, swines, goats, sheep or horses.

The process herein described for developing the Invention Formulation includes the stages below:

a. Stir and mix the esterified carbohydrate with an organic solvent and a vegetable oil

b. Add the hormone together with the oxyethylene polymer, until they dissolve completely

c. Maintain at room temperature

d. Add Ethanol and make up to final volume

In a preferred testing, mix Sucrose Acetate Isobutyrate with sesame oil, and stir at 50° C. Then, add the hormone and Poloxamer 188 and/or Poloxamer 127; stir mixture until complete dissolution of oxyethylene polymer. Maintain at room temperature. Finally, add Ethanol to make up for final volume. In a preferred testing, the hormone is Progesterone and/or Estradiol. In a testing, the organic solvent can be Benzyl Benzoate, Benzyl Alcohol, or mixtures of them.

The Invention Formulation includes an esterified carbohydrate that creates the intra-parenteral depot, which is the microspheres matrix. The carbohydrate can be Sucrose Acetate Isobutyrate (SAIB), Cellulose Acetate Butyrate (CAB), Cellulose Acetate Propionate (CAP), Polycarpolactone, Polylactic Acid, or Polylactic-co-Glycolic Acid (PIGA).

The Invention Formulation includes an organic solvent that contributes to depot division in microspheres. The organic solvent can be selected from the group including vegetable triacetin, isopropyl myristrate, ethyl n-methyl-pyrrolidone oleate, PEG400, Tween 20, Mygliol 812, Imwitor 375, Acetyl Tributyl Citrate (ATBC), Dimethylacetamide (DMA), Glycerol formal, Solutol HS15, Benzyl Benzoate, Benzyl Alcohol, Ethanol, and mixtures of them.

The Invention Formulation includes an oil that helps solvents in separating the microspheres. The oil can be sunflower oil, castor oil, soybean oil, corn oil or sesame oil.

A method for estrous cycle control in mammals is provided upon administration of the Formulations proposed in this invention.

A method for triggering puberty and enhancing pregnancy in mammals is also proposed.

### DRAWINGS DESCRIPTION

FIG. 1 shows photographs taken on an optical 40× microscope; morphological differences in microspheres formed by the solvents can be observed. (Formulation X101) contains Benzyl Alcohol, (Formulation X102) contains Benzyl Benzoate, (Formulation X103) contains Ethanol, (Formulation X104) contains vegetable oil, (Formulation X110) contains a combination of previously mentioned solvents with Pluronic P188, and (Formulation X113) contains a combination of previously mentioned solvents with Pluronic P127.

FIG. 2 indicates in vitro Progesterone release levels, using Formulations X109, X110, X113, X114 and X115.

FIG. 3 details the average Body Condition Scoring (BCS) in a scale from 1 to 5 of the studied animals, divided in the Control Group (“SID Treatment”), and the Group that received the Invention Formulation X115.

FIG. 4 represents the ovarian activity measured by the presence of corpora lutea in animals, which were divided in the Control Group (“SID Treatment”), and the Group treated with the Invention Formulation X115.

FIG. 5 displays the average size of dominant follicles, in centimetres, before observing ovulation, in the Control Group (“SID Treatment”), and the Group treated with the Invention Formulation X115.

FIG. 6 displays the average ovulation moment after administration of Prostaglandin, which is expressed in hours for each group: Control Group (“SID Treatment”) and the Group treated with the Invention Formulation X115.

FIG. 7 shows the number of ovulations and percentage per study group: Control Group (“SID Treatment”) and the Group treated with the Invention Formulation X115.

FIG. 8 shows Progesterone release profiles in bovine serum after an intramuscular injection, after a 12-day treatment, comparing the intra-vaginal device and Invention Formulation X115.

FIG. 9 shows pregnancy percentage achieved for each breed, with the use of 5 ml volume of Formulation X115 versus 0.5 g or 1 g of the intra-vaginal device, depending on the breed.

FIG. 10 shows Progesterone release profile in bovine serum after an intramuscular injection into heifers, during a 12-day treatment. Two different doses (5 ml and 4 ml) of Formulation X115 were administered.

FIG. 11 shows Progesterone release profile in bovine serum after an intramuscular injection into high production Holando cows, during a 12-day treatment. Two different doses (5 ml and 5.5 ml) of Formulation X115 were administered.

FIG. 12 shows Progesterone release profile in bovine serum after an intramuscular injection into Nelore cows, during a 12-day treatment. A 5 ml dose of two different Formulations (X113 and X115) was administered in comparison with a 0.5 g dose of the Progesterone SID intra-vaginal device.

FIG. 13 shows Progesterone release profile in bovine serum after an intramuscular injection into Brangus cows in anestrus, during a 12-day treatment. Two different doses (5 ml and 4 ml) of Formulation X115 were administered.

FIG. 14 shows Progesterone release profile in swine serum after an intramuscular injection into ovariectomized sows, during an 18-day treatment. Two administration schemes were performed: the first included two doses of 5 ml Formulation X115 on Days 0 and 7; and the second included 8 ml on Day 0. Results of both schemes are herein included, as well as the control result using 1 g of intra-vaginal device.

FIG. 15 details the number of hours it took the dominant follicle to ovulate after the administration of Prostaglandin (PG). The comparison was based on breed and ovulation trigger, the latter being Estradiol Benzoate (EB), Equine Corionic Gonadotrophin (eCG) and Estradiol Cypionate (EC).

FIG. 16 indicates the comparative size of dominant follicle in millimetres (mm) on Days 8 and 10 of treatment. The comparison was based on breed and ovulation trigger, the latter being Estradiol Benzoate (EB), Equine Corionic Gonadotrophin (eCG) and Estradiol Cypionate (EC).

FIG. 17 indicates the ovulation percentage by breed and ovulation trigger, the latter being Estradiol Benzoate (EB), Equine Corionic Gonadotrophin (eCG) and Estradiol Cypionate (EC).

FIG. 18 indicates the percentage of heifers not cycling after treatment with Estradiol Benzoate and Formulation X115.

FIG. 19 shows blood Estrogen release curvatures from a simple formulation (X117) against a sustained formulation release (X116).

FIG. 20 shows blood Progesterone release curvatures in two formulations with different amounts of SAIB.

### DETAILED DESCRIPTION OF THE INVENTION

This invention refers to an injectable formulation that is able to release Progesterone for a continuous period of 5 to 9 days, after which it decreases to basal levels, and the administration is synchronized with ovulations. The administered injection produces an intramuscular depot formation system due to the use of esterified carbohydrates, organic solvents, a Progestagenic agent and oxyethylene polymers. The in situ formation of porous microspheres is triggered when the injected solution is exposed to the body fluid medium. Different work protocols were combined to supplement the administration of a hormone system in Nelore and Angus cows, which resulted in 100% ovulations and 60% pregnancies for both sample cases.

Formulation may contain one or more of the different Progesterone solvents, for example, vegetable oils, organic solvents such as Benzyl Alcohol, Benzyl Benzoate and/or Ethanol. It may also include a biodegradable polymer or an esterified carbohydrate with strong water repellency, for example, Sucrose Acetate Isobutyrate (SAIB), Cellulose Acetate Butyrate (CAB), Cellulose Acetate Propionate (CAP), Polycarpolactone, Polylactic Acid, or Polylactic-co-Glycolic Acid (PIGA), that may be suitable for creating intra-parenteral depots; and finally, an oxyethylene polymer, for example, copolymer in Poloxamer block 188, 212, 215, 217, 231, 234, 235, 237, 238, 282, 284, 288, 331, 333, 334, 335, 338, 401, 402, 403 and 407 that, according to their concentration, behave as matrix-forming agents (thixotropic gels capable of retaining different polarity active agents within their polymer structure), as surfactants, or as described later, they behave as polar miscella inside a non-polar system, i.e., they are a pore-forming agent. Formulation may also include antioxidant agents, bactericides, or other excipient.

The Invention Injectable Formulation is a clear liquid solution with a slightly yellow hue, and of parenteral administration, preferably intramuscular. When the Invention Formulation is exposed to a liquid medium in the body, for example, muscles or blood vessels, a local biodegradable depot is created by thousands of microspheres adhered to animal tissue. These particular microspheres show the desired feature of having pores on their surface. These pores improve encapsulated active agent migration to the exterior and water intake, allowing complete release and use of Progesterone for reaching follicular growth, and subsequently, ovulations.

Different combinations between solvents and polymers have been tested. Solvents are compatible with Progesterone, and they do not cause phase precipitation or separation.

A magnetic stirrer and 250 ml beakers were used for all the combinations. Working temperature was 50-60° C., and the mixture was stirred for 15 minutes. Then, it was left to cool until a 25° C. temperature. Stability was studied for 3 months both at room temperature and at fridge temperature.

TABLE 1
Solvents and Polymers Combination

| formulas | SAIB | P188 | P127 | BB | BOH | EtOH | Veg. Oil | Myvacet | DMSO | Glycerol | NMP | P4 | Result at 4° C. | Result at 25° C. |
|---|---|---|---|---|---|---|---|---|---|---|---|---|---|---|
| X101 | 10 | | | | 85 | | | | | | | 5 | homogeneous | homogeneous |
| 8102 | 10 | | | 85 | | | | | | | | 5 | homogeneous | homogeneous |
| X103 | 10 | | | | | 85 | | | | | | 5 | homogeneous | homogeneous |
| x104 | 10 | | | | | | 85 | | | | | 5 | homogeneous | homogeneous |
| x105 | 10 | | | | | | | | | 85 | | 5 | separates | separates |
| x106 | 10 | | | | | | 70 | 15 | | | | 5 | separates | separates |
| x107 | 10 | | | 15 | | 20 | | | | 40 | 10 | 5 | separates | separates |
| x108 | 10 | | | | 25 | 20 | 20 | | 20 | | | 5 | precipitates | precipitates |
| x109 | 10 | | | 19 | 30 | 19 | 17 | | | | | 5 | homogeneous | homogeneous |
| x110 | 10 | 1 | | 19 | 29 | 19 | 17 | | | | | 5 | homogeneous | homogeneous |
| x111 | 10 | 5 | | 19 | 25 | 19 | 17 | | | | | 5 | homogeneous | homogeneous |
| x112 | 10 | | 1 | 19 | 29 | 19 | 17 | | | | | 5 | homogeneous | homogeneous |
| x113 | 10 | | 5 | 19 | 25 | 19 | 17 | | | | | 5 | homogeneous | homogeneous |

As can be noted, the combinations of Formulations X101-X104 and X109-X113 show appropriate results because they do not cause phase precipitation or separation.

Table 1 formulations were left in stability oven at room temperature and at 36° C. for a certain period of time. Then, Progesterone stability was assessed by measuring the remaining quantity.

Table 2 shows the combinations with better Progesterone stability results.

TABLE 2

| | | | | | P4 value | accumulated | accumulated |
|---|---|---|---|---|---|---|---|
| | | | | Original P4 | after 3 weeks | release % | release % |
| formulas | SAIB | EtOH | P4 | value % m/m | at 35° C. | after 24 hours | after 240 hs |
| w101 | 5 | 90 | 5 | 5.2 | 100.4 | 83 | 102 |
| w102 | 12 | 83 | 5 | 4.98 | 100.8 | 78 | 99 |
| w103 | 50 | 45 | 5 | 4.99 | 99 | 65 | 79 |
| w104 | 75 | 20 | 5 | 5.12 | 98.9 | 42 | 62 |

As can be noted from the results shown, the quantity of SAIB adapts perfectly to the expected release profile from Day 1 to Day 10.

### In Situ Formation of Microspheres and Microscopic Characteristics:

After studying Solvents/P4/Polymers System solubility and compatibility features, the resulting microspheres were described by means of adding 5 ml of each formulation to 100 ml of PBS buffer at 25° C.

Formulations tested are described in Table 3.

TABLE 3
microspheres characteristics

| formulas | SAIB | P188 | P127 | BB | BOH | EtOH | Veg. Oil |
|---|---|---|---|---|---|---|---|
| x101 | 10 | | | | 85 | | |
| x102 | 10 | | | 85 | | | |
| x103 | 10 | | | | | 85 | |
| x104 | 10 | | | | | | 85 |
| x110 | 10 | 1 | | 19 | 29 | 19 | 17 |
| x113 | 10 | | 5 | 19 | 25 | 19 | 17 |

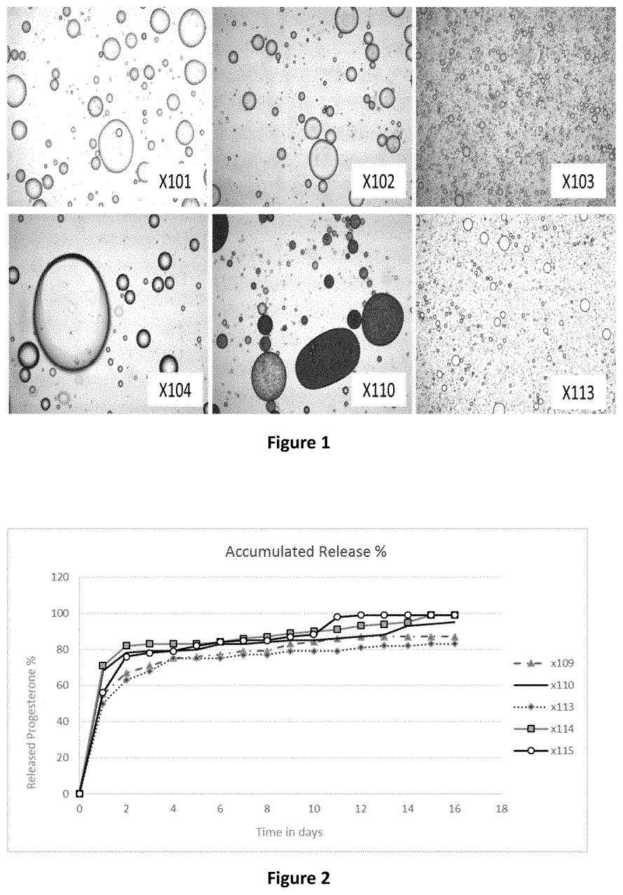

FIG. 1 shows different morphologies and porosity degrees, with the higher porosity present in Formulations X102 and X110.

In a first instance, different concentrations of SAIB and a typical solvent were taken. In subsequent instances, different combinations of solvents (created in advance according to the system stability) were studied, and we proceeded with the study of in vitro Progesterone release.

For in vitro release trials, the formulations detailed in Table 4 were used.

TABLE 4
In vitro release trials:

| formulas | SAIB | P188 | P127 | BB | BOH | EtOH | Veg. Oil | P4 |
|---|---|---|---|---|---|---|---|---|
| x109 | 10 | | | 19 | 30 | 19 | 17 | 5 |
| x110 | 10 | 1 | | 19 | 29 | 19 | 17 | 5 |
| x113 | 10 | | 5 | 19 | 25 | 19 | 17 | 5 |
| x114 | 7 | | | 19 | 33 | 19 | 17 | 5 |
| x115 | 7 | 1 | | 19 | 32 | 19 | 17 | 5 |

FIG. 2 shows release profiles, and it can be noted that after 15 days, 80-100% of Progesterone was released.

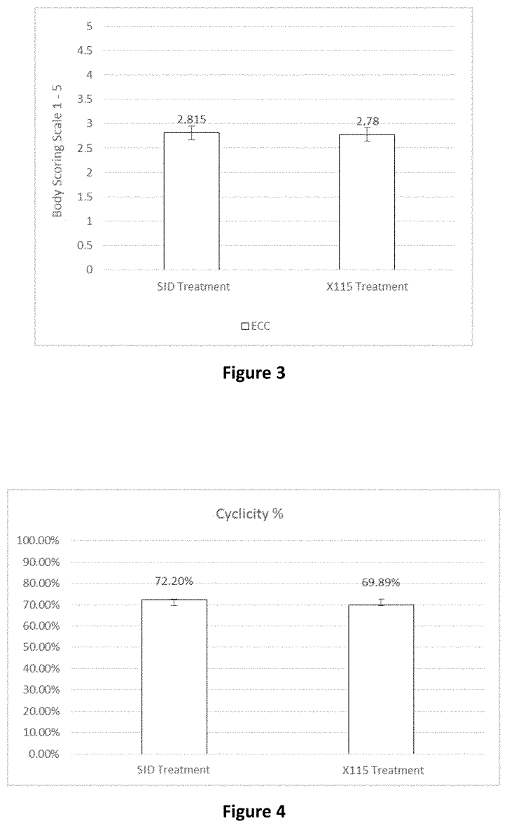

Follicular growth trials and studies were performed, together with ovulation time analysis. We used an Invention Formulation X115 and 39 Angus cows with an average Body Condition Scoring of 2.8±0.4 (scale from 1 to 5), updated health plan, and more than 2 calving. These animals were selected from a larger group. The animal Body Condition Scoring was considered first, and then, its cyclicity. Animals were always fed with grazing alfalfa, and supplemented with corn grains and oat rolls to balance their diet. Animals were divided in 2 groups, as shown in Example 4. The selection criteria for both groups were their similarity in Body Condition Scoring and Cyclicity (see FIGS. 3 and 4).

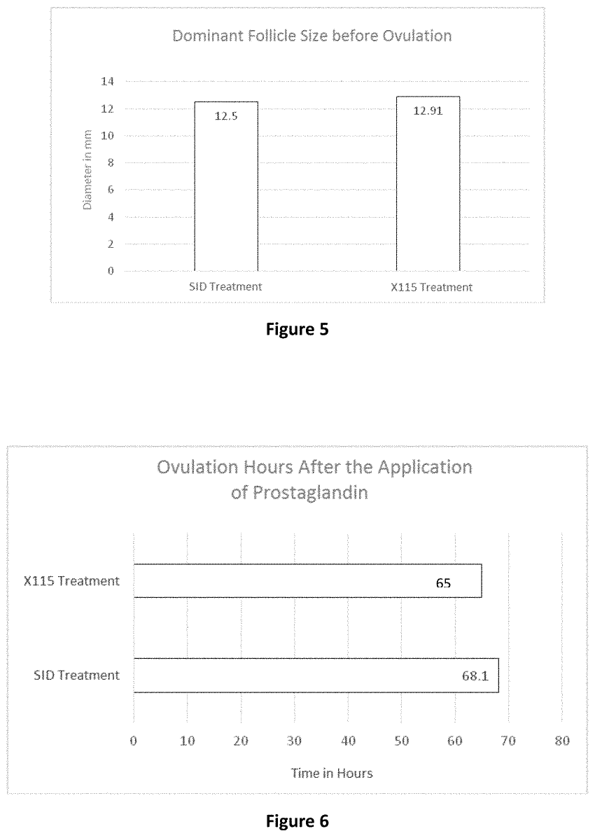

FIG. 5 shows the size of follicles before ovulation for both groups. It can be noted that there are differences in average follicle size at the time of ovulation.

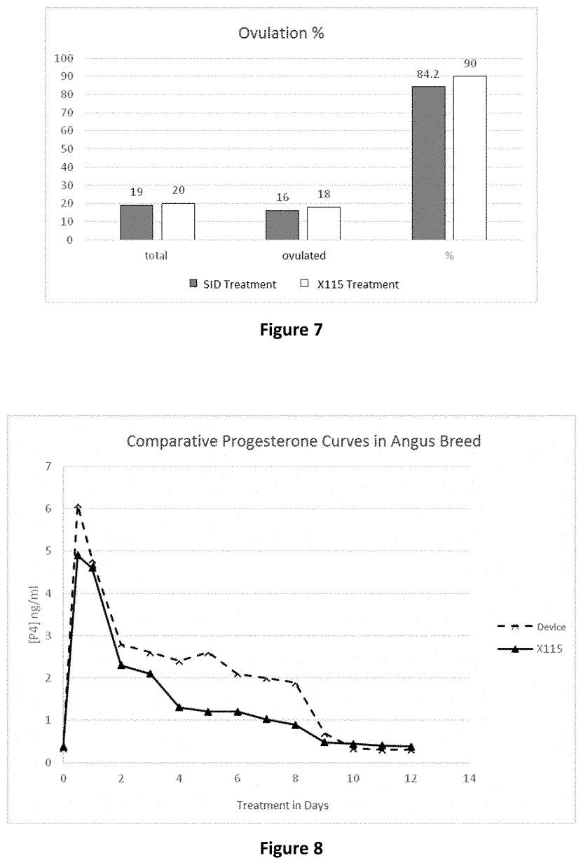

FIGS. 6 and 7 show ovulation percentage and ovulation time for each group. The ovulation percentage is higher in the Study Group versus the Comparison Group.

FIG. 8 shows blood Progesterone concentrations for both groups. As it can be clearly noted, Progesterone concentration remains above 1 ng/ml for over 7 days, a condition that is more than acceptable to prevent advanced ovulations on the 9th or 10th day of treatment. Then, Progesterone decreases to basal levels without having to remove any device.

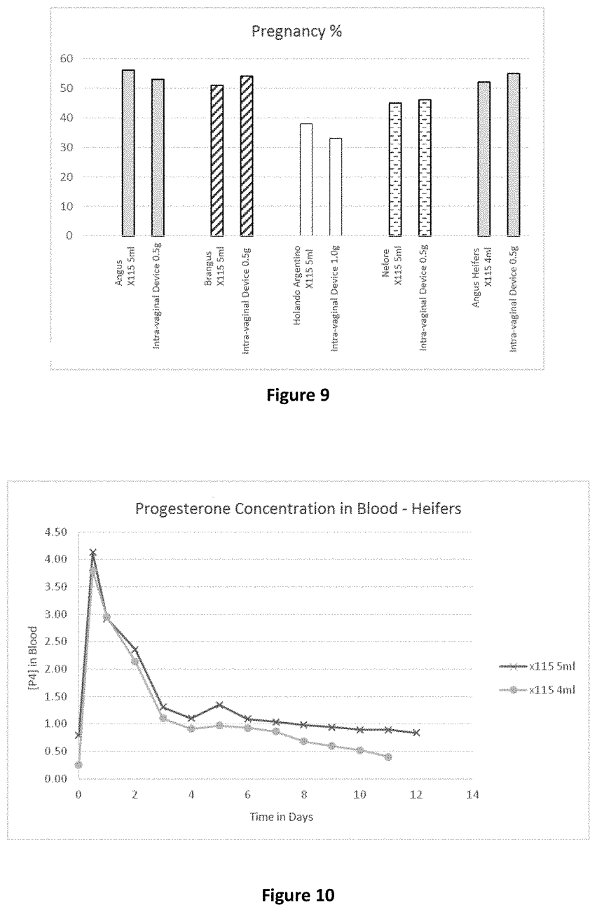

It was then time to do a pregnancy test, for which 210 multiparous Angus cows were selected. These were randomized in two groups of 105 cows each. The approach selected was Protocol described in Example 5. As shown in FIG. 9, results revealed that pregnancy percentages are totally similar to the percentages obtained with the Device, with no significant differences, being 55% and 53%, respectively.

The same protocol, as described in Example 5, was repeated in the following breeds: Angus, Brangus, Holando Argentino, and Nelore; all animals were multiparous cows with Body Condition Scoring between 3 and 3.5. As can be noted from FIG. 9, there are no significant differences in pregnancies among the breeds under study. It is worth highlighting that the results favor Holando Argentino breed. For this reason, it is considered that the product is beneficial and satisfactorily improves operation management and times for an Artificial Insemination at Fixed Time (AIFT).

The level of Progesterone in blood was evaluated by dose adjustment in dairy cows, cebu and hybrid (Braford), at the end of their nutritional anestrus; and in heifers. The main purpose of these trials was to assess the use of injectable Progesterone in different breeds and categories, based on the previous assumption that each category has their particular forms of metabolizing Progesterone.

For heifers, results are detailed in FIG. 10. They show that given the same dose to an animal (5 ml), Progesterone is metabolized differently, resulting in higher values; this is not the case with a 4 ml dose. It can be noted that values decrease to levels below 0.5 ng/ml from Day 9 onwards. These results are possible because the formulation has low viscosity, which allows multi-dose administrations.

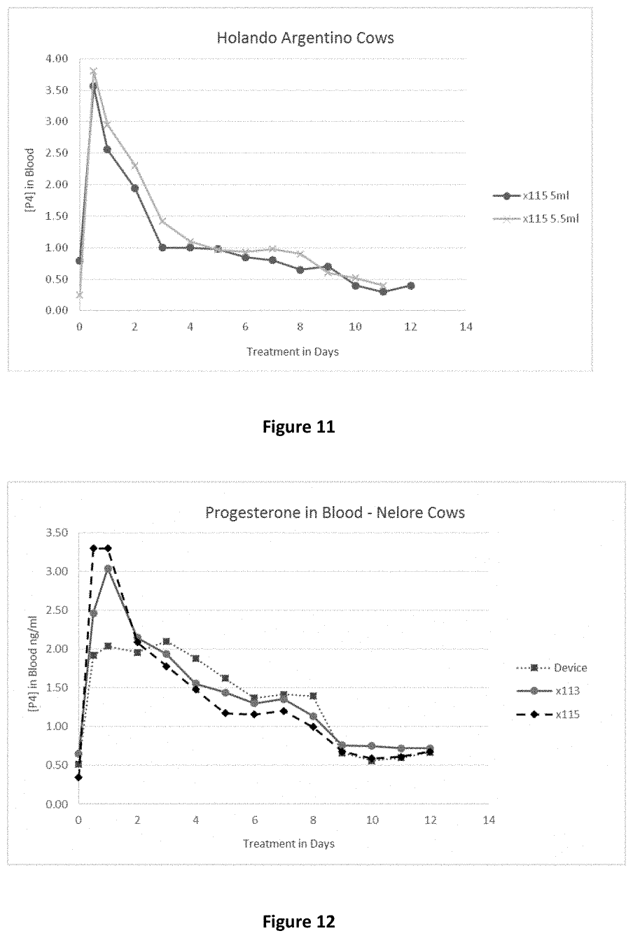

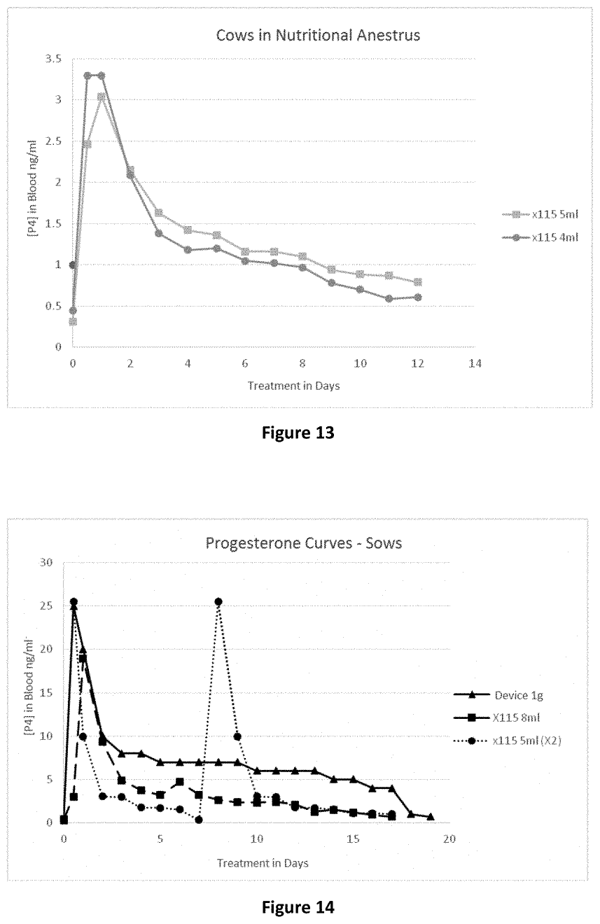

FIGS. 11, 12 and 13 show plasmatic Progesterone level results in Holando, Nelore and British cows in nutritional anestrus. These results evidence formulation flexibility to adapt the Progesterone release kinetics to different bovine breeds and their different metabolic conditions. This, in turn, is an advantage, given the possibility to produce formulations as multi-dose administrations.

Plasmatic Progesterone release trials were performed in sows. As described in Example 7, 12 ovariectomized sows were selected and divided in 3 groups of 4 animals each.

FIG. 14 shows Invention Formulations that can be used in other animal species. For sows, Progesterone requirement had to be maintained for 18 days, instead of 8. The Invention Formulation easily achieved this objective; the only changes were in doses. The formulation may be administered as an 8 ml dose (0.4 g PA). As shown in FIG. 14, the effects of a 1 g Progesterone Device were compared against the Invention Formulation. Progesterone analysis traced a specific curvature that remained constant between 3-1.5 ng/ml, in contrast with the Device, that was between 8-5 ng/ml, with some decreases to basal levels on the same day. It is worth highlighting that the Device blocked animals' physiological response, preventing follicular growth until Day 31, whereas the Invention Formulation triggered growth and subsequent ovulation on Day 19. Therefore, the Formulation is also useful as a replacement of oral treatment and implants in sows.

To confirm ovulation window, several trials on ovulation time were performed in the different bovine breeds. The study groups included 12 animals each. The breeds were: Nelore, Angus and Holando Argentino, and the approach selected was the Protocol described in Table 5 below:

TABLE 5

| | | Day 0 | Day 8 | | Day 9 | | Day 10 | |
|---|---|---|---|---|---|---|---|---|
| type | Enclosures | am | am | pm | am | pm | am | pm |

| | | | | | | | |
|---|---|---|---|---|---|---|---|
| EB wo/eCG | 4 | Estradiol Benzoate: | D,L-Cloprostenol: | | Estradiol | | Al |
| | | 2 ml | 2 ml | | Benzoate: | | |
| | | | | | 1 ml | | |
| | | Injectable Progesterone: | eCG: 300 UI | | | | |
| | | 5 ml | | | | | |
| EB w/eCG | 4 | Estradiol Benzoate: | D,L-Cloprostenol: | | Estradiol | | Al |
| | | 2 ml | 2 ml | | Benzoate: | | |
| | | | | | 1 ml | | |
| | | Injectable Progesterone: | eCG: 300 UI | | | | |
| | | 5 ml | | | | | |
| GnRH | 4 | Estradiol Benzoate: | D,L-Cloprostenol: | | | GnRH: | Al |
| | | 2 ml | 2 ml | | | 2 ml | |
| | | Injectable Progesterone: | eCG: 300 UI | | | | |
| | | 5 ml | | | | | |
| EC wo/eCG | 3 | Estradiol Benzoate: | | D,L-Cloprostenol: | | | Al |
| | | 2 ml | | 2 ml | | | |
| | | Injectable Progesterone: | | Estradiol Cypionato: | | | |
| | | 5 ml | | 1 ml | | | |
| EC w/eCG | 3 | Estradiol Benzoate: | | D,L-Cloprostenol: | | | Al |
| | | 2 ml | | 2 ml | | | |
| | | Injectable Progesterone: | | Estradiol Cypionate: | | | |
| | | 5 ml | | 1 ml | | | |
| | | | | eCG: 300 UI | | | |

(Note:
EB means Estradiol Benzoate and eCG means Equine Corionic Gonadotrophin.)

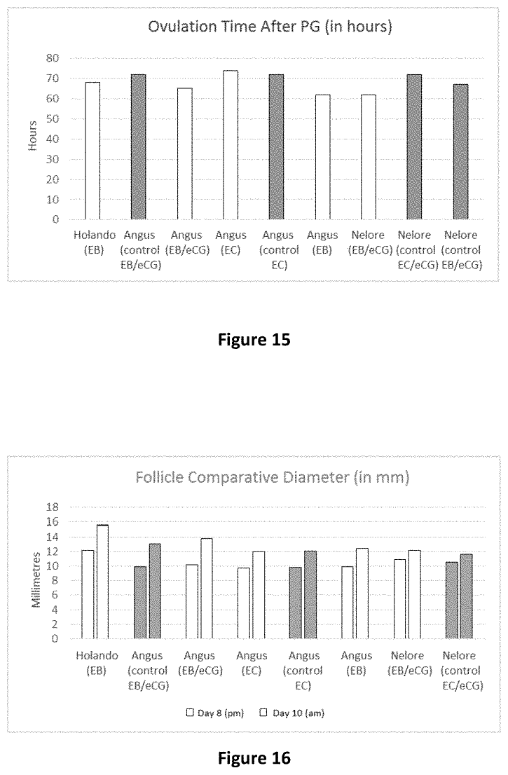

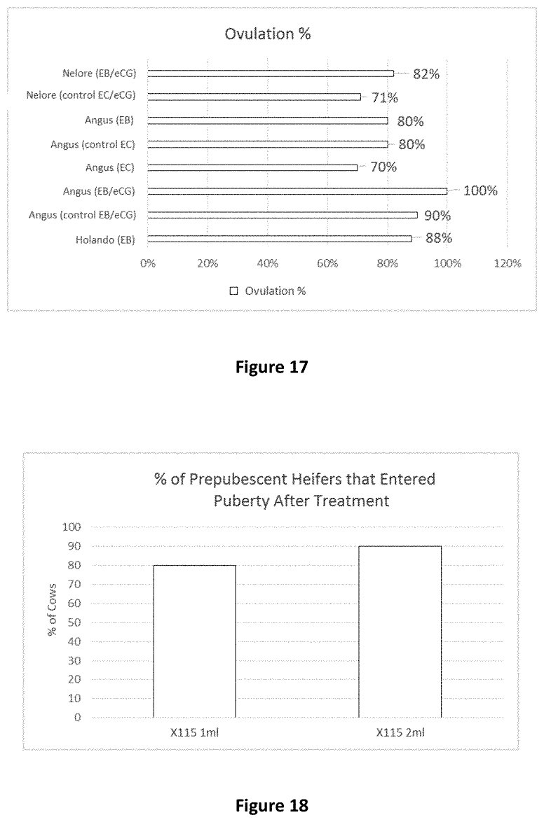

Results can be seen in FIGS. 15, 16 and 17. Despite the fact that there exist certain subtle differences between Protocols and the studied breeds, it can be noted that ovulation percentages and times are similar to those presented by the Devices. Therefore, it can be concluded that it appropriately and accurately relates to the known standard protocols.

The Invention Formulations feature low viscosity (2-6 cP), with a preferred testing of 5 cP at 20° C., what makes them particularly effective for injections. Low viscosity facilitates development of a multi-dose presentation; therefore, for the same presentation of the product, the user may choose different doses of the active ingredient. In this presentation, Formulation is suitable for different breeds or species, for example, bovines, swines, goats, sheep or horses.

Unlike other products, such as thermal gels or emulsions/suspensions; implants or devices; or microspheres formed by solvent evaporation, the development of the Invention Formulation is very simple, it only requires a temperature input of 40-50° C. to mix all the components that, once stirred, become a perfect homogeneous mixture. This is a desired and significant advantage for massive scale production, as it helps reduce labour costs and energy costs, thus, enhancing performance and reducing production downfalls.

As they are injected, the Invention Formulations create a porous and biodegradable three-dimensional structure. This three-dimensional structure is key to cause system collapse and to reduce Progesterone concentration in blood to basal levels, making ovulations and subsequent pregnancies possible. After the ultrasounds that revealed follicular growth, we discovered that pore inducers were necessary as, in their absence, cows did not ovulate and/or presented irregular ovulations, due to late Progesterone decrease, as shown in the results (See FIG. 1 Formulation X115 vs the others, and the tables where the correct Poloxamer is tested upon quantity).

The Invention Formulations provide a controlled release matrix that prevents formulation and pre-formulation inconveniences in the field, providing good control over the estrous cycle. In particular, it allows field formulation, eases tasks, and is accessible for different breeds within the same species, as well as for different species. In fact, it has proved effective in bovine and swine livestock, and it is very promising in sheep. This makes it an essential tool for estrous cycle control in different economic-significant livestock. It is worth highlighting that the Invention Formulations avoid having a product in emulsion and in phases.

Heifers younger than 15 months old are typically prepuberal. It is true that this factor depends on breed, but, generally, Indian breeds, such as Nelore, take longer to enter puberty. In this case, puberty triggering was assessed upon administration of 1 ml or 2 ml of Product X115 (0.05 g and 0.1 g P4), respectively. Results show 80-90% of success for both cases, as can be seen in FIG. 18.

To exemplify, several Formulations are listed, all of them within the scope of the present Invention:

| | Raw materials | % m/m | mg/ml |
|---|---|---|---|
| 1 | BP/USP Progesterone | 5 | 50.0 |
| 2 | Benzyl Alcohol | 32 | 320.0 |
| 3 | Benzyl Benzoate | 19 | 190.0 |
| 4 | Ethanol | 18.7 | 187.0 |
| 5 | Sesame Oil | 18 | 180.0 |
| 6 | Sucrose Acetate Isobutyrate | 4 | 70.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |

| | Raw materials | % m/m | mg/ml |
|---|---|---|---|
| 1 | BP/USP Progesterone | 8 | 80.0 |
| 2 | Benzyl Alcohol | 32 | 320.0 |
| 3 | Benzyl Benzoate | 17 | 170.0 |
| 4 | Ethanol | 15 | 150.0 |
| 5 | Sesame Oil | 17 | 170.0 |
| 6 | Sucrose Acetate Isobutyrate | 7 | 110.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |
| | Total | 100.47 | |

| | Raw materials | % m/m | mg/ml |
|---|---|---|---|
| 1 | BP/USP Progesterone | 4.7 | 47.0 |
| 2 | Benzyl Alcohol | 34 | 340.0 |
| 3 | Benzyl Benzoate | 18 | 180.0 |
| 4 | Ethanol | 15 | 150.0 |
| 5 | Sesame Oil | 17 | 170.0 |
| 6 | Sucrose Acetate Isobutyrate | 7 | 110.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |
| | Total | 100.17 | |

| | Raw materials | % m/m | mg/ml |
|---|---|---|---|
| 1 | BP/USP Progesterone | 5 | 50.0 |
| 2 | Benzyl Alcohol | 34 | 340.0 |
| 3 | Benzyl Benzoate | 18 | 180.0 |
| 4 | Ethanol | 17 | 170.0 |
| 5 | Sesame Oil | 17 | 170.0 |
| 6 | Sucrose Acetate Isobutyrate | 9 | 90.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |
| | Total | 100.47 | |

| | Raw materials | % m/m | mg/ml |
|---|---|---|---|
| 1 | BP/USP Progesterone | 5 | 50.0 |
| 2 | Benzyl Alcohol | 51.5 | 515.0 |
| 4 | Ethanol | 17 | 170.0 |
| 5 | Sesame Oil | 17 | 170.0 |
| 6 | Sucrose Acetate Isobutyrate | 9 | 90.0 |
| | (SAIB) | | |
| 7 | Copolymer in oxy-ethylene-oxy- | 0.37 | 3.7 |
| | propylene block (Pluronic | | |
| | F68/P0loxamer 188) | | |
| 8 | BHT | 0.1 | 1.0 |
| | Total | 99.97 | |

| | Raw materials | % m/m | mg/ml |
|---|---|---|---|
| 1 | BP/USP Progesterone | 5 | 50.0 |
| 2 | Benzyl Alcohol | 68.6 | 686.0 |
| 4 | Ethanol | 17 | 170.0 |
| 6 | Sucrose Acetate Isobutyrate | 9 | 90.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |
| | Total | 100.07 | |

| | Raw materials | % m/m | mg/ml |
|---|---|---|---|
| 1 | BP/USP Progesterone | 5 | 50.0 |
| 2 | Benzyl Alcohol | 51.5 | 515.0 |
| 5 | Sesame Oil | 34 | 340.0 |
| 6 | Sucrose Acetate Isobutyrate | 9 | 90.0 |
| | (SAIB) | | |
| 7 | Copolymer in oxy-ethylene-oxy- | 0.37 | 3.7 |
| | propylene block (Pluronic | | |
| | F68/Poloxamer 188) | | |
| 8 | BHT | 0.1 | 1.0 |
| | Total | 99.97 | |

| | Raw materials | % m/m | mg/ml |
|---|---|---|---|
| 1 | BP/USP Progesterone | 5 | 50.00 |
| 2 | Estradiol Benzoate | 0.04 | 0.40 |
| 3 | Benzyl Alcohol | 32 | 320.0 |
| 4 | Benzyl Benzoate | 19 | 190.0 |
| 5 | Ethanol | 18.7 | 187.0 |
| 6 | Sesame Oil | 18 | 180.0 |
| 7 | Sucrose Acetate Isobutyrate | 7 | 70.0 |
| | (SAIB) | | |
| 8 | Copolymer in oxy-ethylene-oxy- | 0.37 | 3.7 |
| | propylene block (Pluronic | | |
| | F68/Poloxamer 188) | | |
| 9 | BHT | 0.1 | 1.0 |
| | Total | 100.21 | |

The scope of this Invention covers the use of any type of hormone soluble in organic solvents, or mineral or vegetable oils, for example, Progesterone, Estradiol Benzoate, Estradiol Cypionate, D-Cloprostenol Sodium, DL-Cloprostenol Sodium, Deslorelin Acetate, Equine Corionic Gonadotrophin, Follicle-stimulating Hormone, Luteinizing Hormone, Buserelin Acetate and Oxytocin; or Progesterone derivatives, such as Medroxyprogesterone, 17-Hydroxyprogesterone, Medroxyprogesteronte Acetate, Megestrol Acetate, Algesterone, Gestonorone, Ethisterone, Norgestomet, Chlormadinone, Altrenogest, Nomegestrol and Cyproterone Acetate.

Benzyl Alcohol is one of the solvents used to dissolve Progesterone, and an excellent co-solvent of SAIB. It features medium porosity and behaves as a pore inducer. Benzyl Alcohol can be replaced with vegetable triacetin (any combination and any commercial name), isopropyl myristrate, ethyl n-methyl-pyrrolidone oleate, PEG400, Tween 20, Mygliol 812 (or any type of Mygliol), Imwitor 375 (or any type of Imwitor), Acetyl Tributyl Citrate (ATBC), Dimethylacetamide (DMA), Glycerol formal, Solutol HS15, all within the scope of this Invention.

Benzyl Benzoate dissolves Progesterone and SAIB. When it is in contact with water, it helps maintaining microspheres integrity and adherence.

Ethanol is a solvent used to dissolve Progesterone and SAIB. It facilitates the effect of phase inversion quickly, thus favoring spheres or depot formation.

Different natural oils were used, for example, sesame oil that mixes perfectly with Benzyl Benzoate, Ethanol, SAIB, Benzyl Alcohol and Progesterone. Sesame oil can be replaced with sunflower oil, castor oil, soybean oil, corn oil, or any other vegetable oil, all within the scope of this Invention.

SAIB is a modified non-polar polysaccharide that creates depots and, as has been shown in this Administration. SAIB structure is able to capture similar components and it is highly compatible with Progesterone.

Poloxamer 188 is a copolymer in oxy-ethylene-oxy-propylene block. It is amphipathic and features a short chain. In concentrations below its critical miscella conditions, like these, it behaves as a pore inducer and, under certain proportions it creates orifices inside the SAIB microsphere. It can dissolve in water-based or organic media.

Butylated hydroxytoluene (BHT) is an antioxidant agent.

We tested the versatility and comfort of Formulation X116 in this invention, which includes a combination of Estradiol Benzoate and Progesterone to trigger cyclicity and increase pregnancies with bulls (natural mating). Analysis of blood Estradiol and Progesterone levels were performed. Pregnancy was analyzed under natural mating conditions and in animals a few days after delivery. The results showed 45-85% global pregnancies against 33% for control treatments, advancing pregnancies affected by postpartum periods up to 25 days. The result is a clear application aimed at the broader domestic and international market of cows that are not synchronized in AIFT.

The following compositions were developed and produced based on weight %. SAIB (Sucrose Acetate Isobutyrate), P188 (Poloxamer 188), BB (Benzyl Benzoate), BOH (Benzyl Alcohol), EtOH (Ethanol), Veg Oil, Progesterone and Estradiol Benzoate.

| | | | | | | | | | Estradiol |
|---|---|---|---|---|---|---|---|---|---|
| formulas | SAIB | P188 | P127 | BB | BOH | EtOH | Veg. Oil | Progesterone | Benzoate |
| x115 | 7 | 1 | | 19 | 32 | 19 | 17 | 5 | 0 |
| X116 | 5 | 1 | | 21 | 32 | 19 | 17 | 5 | 0.025 |
| X117 | 0 | 0 | 0 | 44 | 23 | 0 | 33 | 0 | 0.1 |

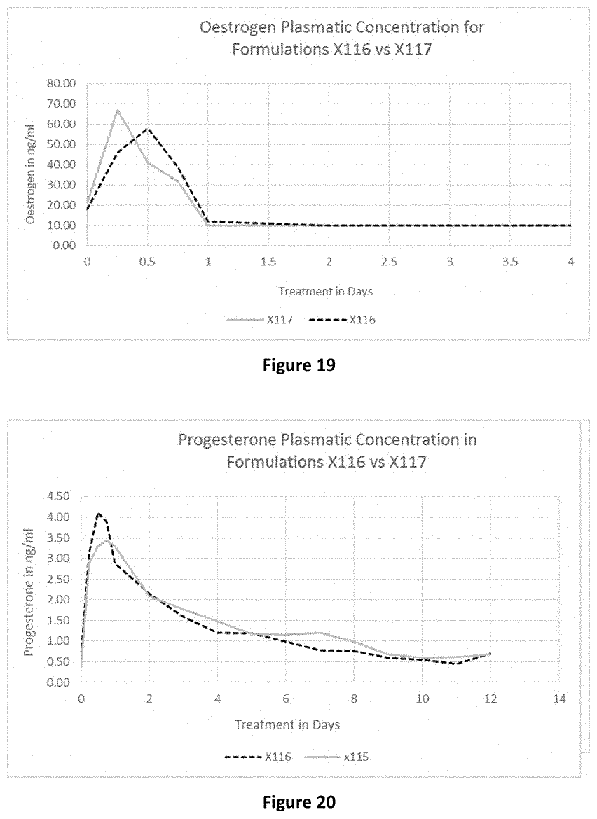

As it can be noted in FIG. 19, Estrogen levels were similar since Day 1 until the administration. Note that for Formulation X116, the amplitude of curvature is greater due to the effect of polymers.

FIG. 20 shows a higher peak with greater slope in the first 2 or 3 days after administration of Formulation X116. This event is the result of both polymer reduction and decrease in the last days of treatment. The administration of Estrogen provided by Formulation X116 is highly functional.

### Triggering Continental Prepuberal Heifers:

Of the total number of females assessed (36), 12 heifers had to be removed from the test because there was evidence of corpus luteum (CL) in the first ultrasound scans. The average weight of females was 378.9±35.85 Kg, and Body Condition Scoring was 3.7±0.8.

| | Estrous | Estrous | Ovulation | |
|---|---|---|---|---|
| | Rate | Time | Rate | DFMT |
| Group treated | 78.6% (11/14) a | 31.6 ± 13.4 | 100% (14/14) a | 14.3 ± 4.2 a |
| with X116 | | | | |
| Control Group | (0/10) b | — | 20% (2/10) b | 16.0 ± 7.1 a |

ab: values with different letters show significant differences.

The administration of Formulation X116 helps reducing cyclic sexual activity in 15-month-old heifers undergoing prepuberal anestrus. This trial supplements the trial described in the paragraph above, thus confirming the advantages of Estradiol Benzoate in the same administration.

### Natural Mating Pregnancy in Prepuberal Heifers

Treatment with Formulation X116 showed the following results:

| | Cows | | |
|---|---|---|---|
| Group | per Group | Pregnancy Percentage | Observations |
| 1 | 101 | 45.5% (45/101) | cyclic heifers |
| 2 | 55 | 33.0% (18/55) | non-cycling heifers |
| 3 | 54 | 0% (0/54) | non-cycling heifers |

It can be observed a difference of 33 points in the groups of cows undergoing anestrus at the time of diagnosis. There is a clear and proven triggering in pregnancies, which advances pregnancies and future deliveries.

### Pregnancy in Suckler Cows Undergoing Postpartum Anestrus

Treatment showed the following results:

| | | Pregnancy Percentage | | | |
|---|---|---|---|---|---|
| | Cows | 40-day | 60-day | 100-day | |
| Group | per Group | ultrasound | ultrasound | ultrasound | Observations |
| 1 | 50 | 4.0% (2/50) | 36% (18/50) | 70% (35/50) | Control batch |
| | | | | | without |
| | | | | | hormones |
| 2 | 104 | 36.5% (38/104) | 72% (75/104) | 92% (96/104) | Batch treated |
| | | | | | with X116 |

There was clear evidence of pregnancy around 22-30 days postpartum. The results of the ultrasound scan performed on Day 40 outnumbered the Control Group in over 32 points. Pregnancies and future deliveries were successfully advanced once more.

Pregnancy in Multiparous Cows with Calf, and Undergoing Nutritional Anestrus Affected by Hormone Dose:

Treatment showed the following results:

| | | Pregnancy Percentage | | | |
|---|---|---|---|---|---|
| | Cows | 45-day | 70-day | 105-day | |
| Group | per Group | ultrasound | ultrasound | ultrasound | Observations |
| 1 | 250 | 43% (108/250) | 56.4% (141/250) | 72.8% (182/250) | 2 ml X116 |
| 2 | 250 | 32% (80/250) | 45% (112/250) | 60.5% (151/250) | 4 ml X116 |
| 3 | 142 | 15% (21/142) | 45.1% (64/142) | 51% (93/142) | Control without |
| | | | | | hormones |

Treatment for cows undergoing deep nutritional anestrus improved their performance after reducing the dose of the administered hormone. This is clear evidence of the product versatility in adjusting the administered dose.

Pregnancy in Multiparous Cows with Calf, with Low Body Condition Scoring Due to AIFT and Prior Progesterone Triggering:

Treatment showed the following results:

| | | Pregnancy Percentage | | | |
|---|---|---|---|---|---|
| | Cows | 45-day | 70-day | 105-day | |
| Group | per Group | ultrasound | ultrasound | ultrasound | Observations |
| 1 | 120 | 62.5% (75/120) | 87.5% (105/120) | 97.5% (117/120) | Pre-triggering |
| | | | | | Group + |
| | | | | | AIFT and |
| | | | | | revision of |
| | | | | | bulls |
| 2 | 130 | 39.2% (51/130) | 63.8% (83/130) | 91.5% (119/130) | AIFT Group |
| | | | | | and revision |
| | | | | | of bulls |
| 3 | 115 | 25.5% (29/115) | 60% (69/115) | 82.6% (95/115) | Control |
| | | | | | without |
| | | | | | hormones |

Group 1 shows a 23.3 points difference in cows pregnancy under ordinary AIFT, and a 37 points difference in the same pregnant cows at the same time, in comparison with the cows that mate with a bull. This confirms that pre-triggering with Formulation X116 improves reproduction techniques via insemination, such as AIFT.

Formulation X116 has been capable of tolerating a combination of two hormones (Estradiol Benzoate and Progesterone) needed for the onset of a follicular wave. Adequate performance was proved by the release kinetics in blood, and by follicle dynamics. Such statements were confirmed by cow pregnancies under various conditions, such as cows with low Body Condition Scoring (deep nutritional anestrus) and cows in recent postpartum.

In such cases, traditional methods did not show the same pregnancy efficiency during the first days of diagnosis. As Formulation X116 is an injectable multi-dose, it simplifies performance and helps determining the exact dosage based on age and nutritional status of the cow. It can easily be administered in big herds, enhancing bull activity, or improving the cow cyclicity percentage.

The present Invention is best illustrated with the Case Studies below, which should not be interpreted as a limitation imposed to its scope. On the contrary, there should be clear understanding of the fact that, after reading the present description, further Invention realizations, modifications and/or equivalents may be suggested by other experts in the field, without deviating from the essence of this Invention or the scope of the Claims attached herein.

### Case Studies

### Example 1—Formulations Development Process

The first instance was to take SAIB to a temperature of 50° C. in its original packaging, and, after reaching said temperature, the desired quantity was weighed and mixed with Benzyl Benzoate, Benzyl Alcohol and oil, stirring at all times and keeping temperature constant until a completely homogeneous mixture was obtained. Later, Progesterone was added together with the oxyethylene polymer. When the polymer was completely dissolved, the sample was left to cool and taken to room temperature. On a later instance, Ethanol was added to the solution to make up for final volume. The result is a 5% P4 clear and transparent solution with a slightly yellow hue.

### Microspheres Formation Assessment:

1 ml of X115 Solution of 5% P4 was introduced in 100 ml distilled water at 30° C. temperature. The event was registered under optical microscope. This effect simulates a test inside the animal body.

### Example 2—Trials on Progesterone In Vitro Release

In a 22×130 mm test tube, we added a phosphate buffered saline (PBS) (10 ml) with a pH of 7.4 or 6.8. The PBS pH (7.4 or 6.8) was determined according to the biologically active substance administration and solubility. The PBS included sodium azide at 0.2% to prevent the growth of microorganisms. Between 0.03 g and 0.09 g of the SAIB/P4/Solvents Formulations were extracted from a disposable plastic Pasteur pipette, and introduced into the test tube; its weight was then registered. Test tubes were closed and placed in a stirrer water bath at 37° C., under constant stirring.

Test tubes were regularly removed. At that time, PBS was removed from the test tube containing the Formulation. These samples were analyzed under ultraviolet radiation to determine the amount of Progesterone present in the PBS solution. Fresh PBS was added to the test tube containing the Formulation, and then, test tube was returned to the stirrer water bath. We repeated this process at different moments.

The concentration of biologically active material in release solutions was used to develop release profiles based on the original quantity of biologically active material in the Formulation. This quantity was determined by high performance liquid chromatography (HPLC) with a diode-array detector (DAD).

### Example 3—Progesterone Release Profiles in Animals

We selected 3 formulations (X113, X114 and X115) to analyze plasmatic levels of Progesterone in blood in comparison with a single 0.5 g Progesterone dose from an Intra-vaginal Device. 40 Angus cows were used, which were divided into 4 groups of 10 animals, with an average Body Condition Scoring of 3 (scale from 1 to 5), updated health plan, and more that 2 calving. Groups were divided according to Progesterone dose, plus the Bovine Intra-vaginal Device Group. Every cow received 2 ml Prostaglandin on Day 8, after 24 and 12 hours, before beginning administration in combination with the insertion of a Crestar implant. This procedure was performed with the intention to eliminate endogenous Progesterone originated in the corpus luteum, which would allow us to obtain a more representative curvature for the plasmatic concentration of exogenous Progesterone. Blood samples were obtained every day, from Day 0 to Day 14. The samples were analyzed by means of particulate electrochemiluminescence, and collected in Vacutainer™ tubes (yellow cap) with coagulation accelerator and gel to separate the serum. Once the sample was extracted, it was left to react at 25° C. for 45 minutes, and then it was precipitated by centrifugation at 3,500 rpm for 15 minutes. Tubes containing separation gel were frozen and thawed at the time of measuring.

### Example 4—Field Efficiency Test

Group 1 (Control): On Day 0, 19 cows received a 0.5 g P4 DIB Device (Syntex) and 2 ml of Estradiol Benzoate (Syntex). On Day 8, the DIB was removed, and the animals received 2 ml of Prostaglandin (Agropharma Cloprostenol), and on Day 9, they received 1 ml of Estradiol Benzoate (Syntex).

Group 2 (Study): On Day 0, 20 cows received 0.5 ml of a selected formulation of Injectable X115 Progesterone (5% P4) and 2 ml of Estradiol Benzoate (Syntex). On Day 8, the animals received 2 ml of Prostaglandin (Agropharma Cloprostenol), and on Day 9, they received 2 ml of Estradiol Benzoate (Syntex).

Diagnosis and Analysis: In order to track follicular dynamics, we performed ultrasound scans on Days 0, 1, 2, 3, 4, 5, 6, 7, 8, 9, 10 am and pm, 11 am and pm, 12 am and pm, 13 am and pm, and 14 am. Ultrasounds were performed with a PIE MEDICAL F100 ultrasound machine, using a 5 mHz transducer. Groups were formed on Day 0 according to cyclicity percentage. We considered “cyclic” those cows that had a corpus luteum (CL) or follicles larger than 8 mm and good uterine tone; and “in surface anestrus” those cows that did not have a corpus luteum or had follicles smaller than 8 mm.

### Example 5—Pregnancy Test

Group 1: On Day 0, 105 cows received a 0.5 g P4 DIB Device (Syntex) and 2 ml of Estradiol Benzoate (Syntex). On Day 8, the DIB was removed, and the animals received 2 ml of Prostaglandin (Agropharma Cloprostenol), and on Day 9, they received 1 ml of Estradiol Benzoate (Syntex).

Group 2: On Day 0, 105 cows received 5 ml of a selected formulation of Injectable X115 Progesterone (5% P4) and 2 ml of Estradiol Benzoate (Syntex). On Day 8, the animals received 2 ml of Prostaglandin (Agropharma Cloprostenol), and on Day 9, they received 2 ml of Estradiol Benzoate (Syntex). 45 days after insemination, the ultrasound scans detected pregnancy.

After this, all Protocols were repeated in different breeds, including Angus, Brangus, Holando Argentino and Nelore. Study subjects included multiparous females with Body Condition Scoring 3-3.5, and 24-month-old nulliparous Angus heifers with Body Condition Scoring 3. The trials were performed in 100 animals per group, in comparison with the 0.5 g Intra-vaginal Device, except in the case of Holando Argentino cows, for which 1 g Progesterone Devices were used.

### Example 6—Analysis of Blood Progesterone by Dose Adjustment in Dairy Cows, Cebu and Hybrid (Braford), at the End of their Nutritional Anestrus, and in Heifers

First Trial: We selected 34 18-month-old nulliparous Angus heifers with Body Condition Scoring 3.2 (on average), and updated health plan. They were divided into two groups, which were administered 5 ml of a selected formulation of X115 (250 mg Progesterone), and then, 4 ml of same formulation (200 mg Progesterone). Every animal received 2 ml Prostaglandin on Day 8, after 24 and 12 hours, before beginning administration in combination with the insertion of a Crestar implant. This procedure was performed with the intention to eliminate endogenous Progesterone originated in the corpus luteum, which would allow us to obtain a more representative curvature for the plasmatic concentration of exogenous Progesterone. Blood samples were obtained every day, from Day 0 to Day 14. The samples were analyzed by means of particulate electrochemiluminescence, and collected in Vacutainer™ tubes (yellow cap) with coagulation accelerator and gel to separate the serum. Once the sample was extracted, it was left to react at 25° C. for 45 minutes, and then it was precipitated by centrifugation at 3,500 rpm for 15 minutes. Tubes containing separation gel were frozen and thawed at the time of measuring.

Second Trial: We selected 26 Holando Argentino cows with a Body Condition Scoring 3.0 and 64 days after giving birth. Average dairy production was 38 L/day during the 10 days of treatment. The approach applied was the Work Protocol previously described. In this trial, two doses of injectable Progesterone of Formulation X115 were studied: one 5 ml Progesterone dose, and one 5.5 ml Progesterone dose (250 mg PA and 275 mg PA, respectively).

Third Trial: We selected 45 multiparous Nelore cows with a Body Condition Scoring 2.8. Two Formulations (X115 and X113) were analyzed. The Protocol applied was the same protocol described for the First Trial. A group of 22 animals was administered 5 ml (250 mg P4) of Formulation X113, whereas a group of 23 animals was administered 5 ml (250 mg P4) of Formulation X115.

Fourth Trial: We selected 30 Braford cows with Body Condition Scoring 2, at the end of their nutritional anestrus due to flooding. The Protocol applied was the same protocol described for the First Trial. The animals were divided into two groups of 15 cows each, and were administered 5 ml of Formulation X115 and 4 ml of Formulation X115.

Fifth Trial: We selected 200 heifers younger than 15 months old. We performed ultrasound scans to verify the absence of ovarian activity, understood as the absence of corpora lutea or follicles larger that 10 mm in diameter. They were divided into two groups. The First 100 animals Group was administered 1 ml of Formulation X115 (50 mg PA) plus 2 ml Estradiol Benzoate (2 mg Estrogen), whereas the Second 100 animals Group was administered 2 ml of Formulation X115 (100 mg PA) plus 2 ml Estradiol Benzoate (2 mg Estrogen). After 21 days, the ultrasound scan was repeated and we noticed the presence of corpora lutea.

### Example 7—Plasmatic Progesterone Release Trials in Sows

For this trial, we selected 12 ovariectomized sows that were divided into 3 groups of 4 animals each. The animals are nulliparous pubescent females (N=12), weighing approximately 90-100 Kg. Five days after their arrival and a period of acclimatization, the sows underwent minor surgery to remove their ovaries (ovariectomy). After surgery, animals were given a post-operative recovery period of 5 days. Sows were divided into 3 groups. The First Group was administered a 1 mg Progesterone Intra-vaginal Device; the Second Group was administered an 8 ml injection of Formulation X115; and the Third Group received two administrations of 5 ml Formulation X115, separated by a 7-day interval. With the aim to verify Progesterone serum levels, blood samples were extracted from each animal, once per day, from the day before administering Progesterone (D1) until the day after removal (D15).

### Example 8—Analysis of Ovulation Time in Different Breeds Regarding Protocol Effect in Comparison with an Intra-Vaginal Device

To confirm ovulation window, several trials on ovulation time were performed in different bovine breeds. We worked with groups of 12 animals from different bovine breeds, including Nelore, Angus and Holando Argentino. Protocols detailed in Table 5 above were applied.

### Example 9—Estrogen and Plasmatic Progesterone Measurements in Combined Formulas Versus Individual Formulas

In order to track follicular dynamics, we performed ultrasound scans on Days 0, 4, 6, 8, 9, 10 am and pm, 11 am and pm, 12 am and pm, 13 am and pm, and 14 am. Ultrasounds were performed with a PIE MEDICAL F100 ultrasound machine, using a 5 mHz transducer. Groups were formed on Day 0 according to cyclicity percentage. We considered “cyclic” those cows that had a corpus luteum (CL) or follicles larger than 8 mm and good uterine tone; and “in surface anestrus” those cows that did not have a corpus luteum or had follicles smaller than 8 mm.

We selected 34 multiparous Braford heifers with a Body Condition Scoring 3.4 (on average), and updated health plan. They were divided into two groups. The first group received 4 ml of a selected formulation of X115 (200 mg Progesterone), and then, 2 ml of formulation X117 (2 mg Estradiol Benzoate) (Estradiol Benzoate Composition). The second group incorporated formulation X116. Every animal was administered 2 ml Prostaglandin on Day 8, after 24 and 12 hours, before beginning administration of every Progesterone and Estrogen administration, in combination with the insertion of a Crestar implant. This procedure was performed with the intention to eliminate endogenous Progesterone originated in the corpus luteum, which would allow us to obtain a more representative curvature for the plasmatic concentration of exogenous Progesterone. Blood samples were obtained on a daily basis, from Day 0 to Day 14, excepting Day 1 when samples were extracted every 6 hours. The samples were analyzed by means of particulate electrochemiluminescence, and collected in Vacutainer™ tubes (yellow cap) with coagulation accelerator and gel to separate the serum. Once the sample was extracted, it was left to react at 25° C. for 45 minutes, and then it was precipitated by centrifugation at 3,500 rpm for 15 minutes. Tubes containing separation gel were frozen and thawed at the time of measuring.

### Example 10—Triggering Continental Prepuberal Heifers

We worked in the south area of the Province of Cordoba (Argentina), with 36 Charolais breed heifers, 15-16 months old and an average weight of 385 Kg. A portable Honda ultrasound scan was used with a 5 mHz convex transducer. Heifers were divided into two groups: (i) with evidence of ovarian activity, and (ii) without evidence of follicles or corpora lutea. They were later divided into two groups: 1) with treatment on Day 0 of 4 ml Formulation X117 and on Day 8 of 2 ml Estradiol Benzoate, whereas group 2) was Control Group without any hormone administration. On Days 8 and 9, we measured estrous rate, and on Day 18 we confirmed ovulation upon presence of corpus luteum.

### Example 11—Natural Mating Pregnancy in Prepuberal Heifers

This trial was performed in the Province of La Pampa (Argentina), in 210 Red Angus breed heifers, 14-16 months old and a Body Condition Scoring 3.2. A portable Honda ultrasound scan was used with a 5 mHz convex transducer. Heifers with evidence of ovarian activity and evidence of big follicles or corpora lutea were assigned to Group 1), and separated from the heifers with no evidence of follicular activity. The latter were subsequently divided in two subgroups: 2) and 3), where Subgroup 2) was administered 2 ml of Formulation X116 and Subgroup 3) was Control Group without hormone treatment. Bulls were released immediately after group division. On the 45th day, we performed an ultrasound scan to confirm pregnancies.

### Example 12—Pregnancy in Suckler Cows Undergoing Postpartum Anestrus

This trial was performed in the Province of Córdoba (Argentina), in 154 Black Angus breed heifers, with a Body Condition Scoring 3.0 and 26-31 after delivery. A portable Honda ultrasound scan was used with a 5 mHz convex transducer. Absence of follicular activity was then confirmed. Heifers were subsequently divided into two randomized groups: Group 1) was administered 4 ml of Formulation X116, and Group 2) was Control Group without hormone treatment. Bulls were released 4 days after group division. 40, 60 and 100 days after bull releasing, we performed ultrasound scans for pregnancies to assess the results.

### Example 13—Pregnancy in Multiparous Cows with Calf, and Undergoing Nutritional Anestrus Affected by Hormone Dose

This trial was performed in the Province of Corrientes (Argentina), in 542 Black Braford breed heifers, with a Body Condition Scoring 2.2 and 60 days after delivery. A Chison DP10 ultrasound scan was used with a 5 mHz convex transducer to analyze pregnancies. Previously, heifers were divided into three randomized groups: Group 1) was administered 2 ml of Formulation X116 on Day 0 of Protocol, and 2 ml of Estradiol Benzoate on Day 8; Group 2) was administered 4 ml of Formulation X116 on Day 0, and 2 ml of Estradiol Benzoate on Day 8; and Group 3) was Control Group without hormone treatment. Bulls were released 2 days after group division. 45, 70 and 105 days after bull releasing, we performed ultrasound scans for pregnancies to assess the results.

### Example 14—Pregnancy in Multiparous Cows with Calf, with Low Body Condition Scoring Due to AIFT and Prior Progesterone Triggering

This trial was performed in the Province of Cordoba (Argentina), in an end batch (cows that gave birth later in the year) of 375 Brangus breed cows, with a Body Condition Scoring 2.5, and around 60 days after delivery. A PIE MEDICAL F100 ultrasound scan was used with a 5 mHz convex transducer to analyze pregnancies. Previously, cows were divided into three groups. Group 1) was administered 4 ml of Formulation X221 on Day −12 of Protocol. Then, on Day 0, we performed an AIFT, administering 2 ml of Estradiol Benzoate and introducing a single use intravaginal device of 0.5 g Progesterone on Day 0. On Day 8, 2 ml Prostaglandin were administered together with 1 ml Estradiol Cypionate, inseminating on Day 10 at 9 am. Group 2) was subjected to an AIFT without triggering; we administered 2 ml Estradiol Benzoate and introduced a single use intravaginal device of 0.5 g Progesterone on Day 0. On Day 8, 2 ml Prostaglandin were administered together with 1 ml Estradiol Cypionate, inseminating on Day 10 at 9 am. Group 3) was Control Group without any hormone treatment. Bulls were released 12 days after group division for Group 3), and after 30 days for Groups 1) and 2). The number of bulls was 1 every 25 cows, for all three groups. Finally, 45, 60 and 95 days after the AIFT for Groups 1) and 2), and 12 days after releasing the bulls for Group 3) we performed ultrasound scans for pregnancies.

The nature and embodiments of the invention in which an exclusive property or privilege is claimed are defined as follows:

1. A hormone injectable formulation which comprises a hormone, an esterified carbohydrate, organic solvents, vegetable oils, oxyethylene polymers and excipients, where the formulation forms porous microspheres when in contact with a water-based liquid medium, and where the formulation is a single-phase clear liquid.

2. The Formulation described in claim 1 is characterized by selecting the hormone from the group consisting of Progesterone, Estradiol, Estradiol Benzoate, Estradiol Cypionate, D-Cloprostenol Sodium, DL-Cloprostenol Sodium, Deslorelin Acetate, Equine Corionic Gonadotrophin, Follicle-stimulating Hormone, Luteinizing Hormone, Buserelin Acetate and Oxytocin.

3. The Formulation described in claim 1 is characterized by the hormone, which is Progesterone, and is selected from the group comprising Medroxyprogesterone, 17-Hydroxyprogesterone, Medroxyprogesteronte Acetate, Megestrol Acetate, Algesterone, Gestonorone, Ethisterone, Norgestomet, Chlormadinone, Altrenogest, Nomegestrol and Cyproterone Acetate.

4. The Formulation described in claims 1 and 3 is characterized by the additional incorporation of Estradiol.

5. The Formulation described in claim 4 is characterized by the Estradiol, which is Estradiol Benzoate.

6. The Formulation described in claim 1 is characterized by the esterified carbohydrate, which is selected from the group consisting of Sucrose Acetate Isobutyrate (SAIB), Cellulose Acetate Butyrate (CAB), Cellulose Acetate Propionate (CAP), Polycarpolactone, Polylactic Acid, or Polylactic-co-Glycolic Acid (PIGA).

7. The Formulation described in claim 1 is characterized by the organic solvent, which is selected from the group consisting of vegetable triacetin, isopropyl myristrate, ethyl n-methyl-pyrrolidone oleate, PEG400, Tween 20, Mygliol 812, Imwitor 375, Acetyl Tributyl Citrate (ATBC), Dimethylacetamide (DMA), Glycerol formal, Solutol HS15, Benzyl Benzoate, Benzyl Alcohol, Ethanol, and mixtures of them.

8. The Formulation described in claim 1 is characterized by the vegetable oil, which is selected from the group consisting of sunflower oil, castor oil, soybean oil, corn oil, or sesame oil.

9. The Formulation described in claim 1 is characterized by the oxyethylene polymer, which is selected from the group consisting of Poloxamer 188 and Poloxamer 127.

10. The Formulation described in claim 1 is characterized by its concentration, which is as follows: 50-80 mg/ml Progesterone, 50-120 mg/ml Sucrose Acetate Isobutyrate, 900-190 mg/ml Ethanol, 850-190 mg/ml Benzyl Benzoate, 850-190 mg/ml Benzyl Alcohol, 850-170 mg/ml vegetable oil, and 50-3 mg/ml of at least one oxyethylene polymer, selected from the group consisting of Poloxamer 188 or Poloxamer 127.

11. The Formulation described in claim 1 is characterized by its viscosity, which consists of levels between 2-6 cP at a 20° C. temperature.

12. The Formulation described in claim 1 is characterized by the mammal, which is selected from the group comprising bovines, swines, goats, sheep and/or horses.

13. The Formulation described in claim 5 is characterized by its concentration, which is as follows: 50-80 mg/ml Progesterone, 0.040-0.025 Estradiol, 50-120 mg/ml Sucrose Acetate Isobutyrate, 900-190 mg/ml Ethanol, 850-190 mg/ml Benzyl Benzoate, 850-190 mg/ml Benzyl Alcohol, 850-170 mg/ml vegetable oil, and 50-3 mg/ml of at least one oxyethylene polymer, selected from the group consisting of Poloxamer 188 or Poloxamer 127.

14. A process for developing the formulation described in claim 1, which is characterized by the stages below:
a. Stir and mix the esterified carbohydrate with an organic solvent and a vegetable oil
b. Add the hormone together with the oxyethylene polymer, until they dissolve completely
c. Maintain at room temperature
d. Add Ethanol and make up to final volume

15. The Process described in claim 14 is characterized by Stage a., which is developed at a 50° C. temperature, and where the esterified carbohydrate is Sucrose Acetate Isobutyrate, and the vegetable oil is sesame oil.

16. The Process described in claim 14 is characterized by Stage b., in which the oxyethylene polymer is selected from the group consisting of Poloxamer 188 or Poloxamer 127, and this mixture is stirred until complete dissolution of the oxyethylene polymer.

17. The Process described in claim 14 is characterized by the hormone, which is selected from the group consisting of Progesterone, Estradiol, and combinations of both hormones.

18. The Process described in claim 14 is characterized by the organic solvent, which is selected from the group consisting of Benzyl Benzoate, Benzyl Alcohol, and mixtures of them.

19. A method for controlling estrous cycle in mammals characterized by the administration of a determined quantity of Formulation described in claim 1.

20. The method described in claim 19 is characterized by the addition of Estradiol Benzoate and Prostaglandin.

21. A method to trigger puberty and improve pregnancy in mammals that is characterized by its administration, in which the cows are administered a determined amount of the Formulation described in claim 4.

22. The method described in any claim from 19 to 21 is characterized by the mammal, which is selected from the group comprising bovines, swines, goats, sheep and horses.
